(19) United States
(12) Patent Application Publication
(10) Pub. No.: US 2006/0173429 A1
(43) Pub. Date: Aug. 3, 2006

(54) Absorbent article with logically correlated image and textual graphics

(76) Inventor: Deanna M. Acors, Limerick, PA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/088,878

(22) Filed: Mar. 25, 2005

Related U.S. Application Data
(63) Continuation-in-part of application No. 11/047,628, filed on Feb. 2, 2005.

(51) Int. Cl. A61F 13/15

(52) U.S. Cl. 604/361

(57) ABSTRACT

A graphic image for absorbent articles is provided which includes an image graphic and a textual graphic. The image graphic depicts an activity, state or condition and the textual graphic is a word, phrase, sentence or command that describes the activity, state or condition and is logically correlated to the image graphic.

This application is a Continuation-In Part of U.S. patent application Ser. No. 11/047,625 filed Feb. 2, 2005, entitled “Absorbent Article With Logically Correlated Image And Textual Graphics,” the contents of which are incorporated herein in their entirety to the extent that it is consistent with this invention and application.

### FIELD OF THE INVENTION

The present invention relates to absorbent articles and methods of manufacturing absorbent articles and more particularly to absorbent articles having logically correlated image and textual graphics for use as educational and motivational aids for the wearer of the article.

### BACKGROUND OF THE INVENTION

Disposable absorbent articles, including child diapers, training pants, pull-up pants, nighttime incontinence pants and swimming protection garments are known in the art. Though different in appearance and dimensions, all of these products perform the same basic function of fluid and waste absorption/retention and are all based on the same basic design. Nearly all absorbent articles are comprised of a top sheet, a back sheet, and an absorbent core sandwiched between the top sheet and back sheet and located on the article in the crotch region. Optionally, a fluid acquisition layer may also be disposed between the top sheet and the absorbent core. The acquisition layer, for example, acquisition fibers, usually is incorporated in the absorbent articles to provide better distribution of liquid, increased rate of liquid absorption, reduced gel blocking, and improved surface dryness. A wide variety of acquisition fibers are known in the art. Included among these are synthetic fibers, a composite of cellulosic fibers and synthetic fibers, and cross-linked cellulosic fibers. Rather than acquisition fibers, the acquisition layer may be an apertured film such as is disclosed in U.S. Pat. No. 6,610,391, the contents of which are hereby incorporated by reference in their entirety.

Use of disposable absorbent articles in emerging and developed countries is now nearly universal. Most parents and/or caregivers use these products from birth until their children are completely toilet trained. As evidence of this, absorbent children's diapers are now available in a range of styles and sizes ranging from infant diapers, toddler diapers from 1T to 4T, pull-up-style garments and even nighttime bed wetting underwear-style garments sized for children in excess of 85 pounds. Grocery stores and other retail outlets typically dedicate a significant amount of retail space to display the variety of different sized absorbent article products. As a result of the tremendous demand for these products, significant resources have been expended on making functional and aesthetic improvements to them.

Toilet training of infants is a time consuming and at times challenging process for both the child and the caregiver. Various methods have been employed to facilitate this process such as books, charts, potty chairs, and pull-up style diaper designs. Pull-up style diapers come pre-fastened and are pulled up like a pair of underwear and pulled down to facilitate toilet use. This is to help reinforce to the child that he or she may now use the toilet like an older child or an adult. In order to enhance the experience for both the child wearing the absorbent article, as well as the caregiver charged with changing and/or providing articles for the child, absorbent articles often include one or more graphic images designed to generate pleasing and reinforcing reactions from the wearer. These graphic images can be cartoon images, simple shapes, licensed characters, colors, scenes, objects, animals, characters or images from movies, books and/or television shows familiar to the child, anthropomorphic characters engaged in a variety of humanistic activities and various other images. Examples of anthropomorphic characters include dogs, cats, birds, bears, zoo animals, etc. These anthropomorphic characters are often depicted with humanoid facial expressions, having humanoid postures and abilities, engaged in humanoid activities and wearing clothing.

Typically, these graphic images are either printed directly on a poly sheet component of the outer garment layer of the absorbent article, or they are printed on another strip that is adhered or otherwise attached to or visible from the outer front and, optionally rear, portion of the article. Several U.S. Patents disclose absorbent articles including graphic images manufactured according such methods. For example, U.S. Pat. No. No. 4,249,532, which is hereby incorporated by reference, discloses graphics that are pre-printed on a poly sheet used as an outer layer of the absorbent article. U.S. Pat. No. 6,342,528, which is hereby incorporated by reference, discloses an absorbent article having hidden graphics that are revealed only when the fastening strips are opened.

There are numerous reasons for using graphic characters on absorbent articles. Their use may be intended to introduce an element of entertainment or diversion for the child to the otherwise intimidating process of toilet training. They may also distract the child and reduce anxiety and discomfort during diaper changes by providing a familiar and pleasing character to the child. Graphic characters can also influence purchasing decisions made by parents/caregivers as well as personal preferences of the child based on familiarity with and affinities for particular characters.

More recently, active or dynamic graphics have been incorporated into absorbent articles, such as, for example, as disclosed in U.S. Pat. Nos. 6,635,797 and 6,710,221, the contents of which are hereby incorporated by reference. Active graphics are graphics that either appear or disappear when exposed to fluid. As a result, these active graphics are often referred to as wetness indicators. Wetness indicator graphics are usually small objects located in the crotch region of an absorbent article. When a child insults the absorbent articles, the wetness indicators either appear or disappear depending on the type used. Their appearance or absence serves as an indicator to the care giver that the absorbent article has been soiled and needs to be changed.

Though wetness indicators may indicate when a diaper has been urinated in, they serve primarily as a tool for caregivers, as opposed to a learning tool for children wearing the absorbent articles. Because children frequently utilize diapers/training pants through 3 years of age and older, and nighttime training pants even years beyond that, absorbent article graphics provide long term opportunities not only to entertain children and assist in the toilet training process, but also to educate and induce particular behaviors in children. Until now, it is not believed that absorbent article graphics have maximized the opportunity to encourage, entertain and educate children during the diapering process.

The description herein of various advantages and disadvantages associated with known apparatus, methods, and materials is not intended to limit the scope of the invention to their exclusion. Indeed, various embodiments of the invention may include one or more of the known apparatus, methods, and materials without suffering from their disadvantages.

### SUMMARY OF THE INVENTION

Therefore, it would be desirable to provide an absorbent article having a graphic image and a textual graphic which is logically correlated to that image.

It would also be desirable to provide a graphic image for use with absorbent articles that teaches character, activity and word recognition. An additional desirable feature would be-to provide a graphic image for absorbent articles that provides improved entertainment and educational benefits to the wearer. Yet another desirable feature would be to provide an absorbent article that depicts images of characters or scenes that are designed to induce a particular behavior by a wearer of the article, along with a textual graphic that is logically correlated to the image graphic.

In one preferred embodiment of this invention, an absorbent article has a graphic image of a character performing an activity and a textual graphic which is logically correlated to that activity. It is an additional feature of an embodiment of this invention to provide a graphic image for use with absorbent articles that teaches both character and word recognition. An additional feature of this invention is to provide a graphic image for absorbent articles that provides improved educational benefits to the wearer.

In another preferred embodiment, an absorbent article includes an image graphic depicting a condition or state, such as the weather outside, the mood, temperament, or thought of a character graphic, the time of day, or the like, along with a textual graphic that is logically correlated to the image graphic. In some, but not all instances, an image graphic depicting a state or condition is directly logically correlated to the textual graphic, such as a scene of children building a sand castle on a sunny day at the beach along with a textual graphic saying “Fun and sun at the beach.” In other cases, the image graphic depicts a scene or condition that is logically correlated to a textual graphic, such as a scene of a moon and stars and along with a textual graphic containing an expression such as only the letters “Zzzz” indicating sleeping.

In one embodiment of this invention, an absorbent article is provided. The absorbent article according this embodiment comprises a top sheet layer, a back sheet layer, an absorbent core provided at least partially between the top sheet layer and the back sheet layer, and a graphic image located on a portion of the back sheet layer, the image graphic depicting an activity, state or condition and a textual graphic wherein the textual graphic is logically correlated to the activity, state or condition depicted in the image graphic.

In another embodiment according to this invention, a graphic image is disposed so as to be visible on an outer facing surface of an absorbent article. The graphic image according to this embodiment depicts an activity, state or condition and a textual graphic wherein the textual graphic is logically correlated to the activity, state or condition depicted in the image graphic.

In yet a further embodiment according to this invention, a method of manufacturing an absorbent article having a graphic image is provided. The method according to this embodiment comprises registering a graphic image so as to be visible on an outer facing surface of the absorbent article, including registering a graphic image depicting an activity, state or condition and a textual graphic wherein the textual graphic is logically correlated to the activity, state or condition depicted in the image graphic.

These and other features and advantages of the preferred embodiments will become more readily apparent when the detailed description of the preferred embodiments is read in conjunction with the attached drawings.

### BRIEF DESCRIPTION OF THE DRAWINGS

FIG. 1 is a perspective schematic view of a conventional absorbent article having an absorbent core in accordance with various exemplary embodiments of this invention;

FIG. 2 is a perspective view of a pull-up style absorbent article employing conventional graphic images including both permanent and active graphic images;

FIG. 3 is a perspective view of a pull-up style absorbent article employing conventional graphic images depicting a variety of objects and textual descriptions of each object;

FIG. 4 is a perspective view of a pull-up style absorbent article employing graphic images in accordance with various embodiments of this invention;

FIG. 5 is a perspective view of another pull-up style absorbent article employing graphic images in accordance with various embodiments of this invention;

FIG. 6 is a perspective view of yet another pull-up style absorbent article employing graphic images in accordance with various embodiments of this invention;

FIG. 7 is a perspective view of an additional pull-up style absorbent article employing graphic images in accordance with various embodiments of this invention;

FIG. 8 is a perspective view of still a further pull-up style absorbent article employing graphic images in accordance with various embodiments of this invention; and

FIG. 9 is a perspective view of yet an additional pull-up style absorbent article employing graphic images in accordance with various embodiments of this invention.

### DETAILED DESCRIPTION OF THE INVENTION

The following description is intended to convey a thorough understanding of the invention by providing specific embodiments and details involving absorbent articles with graphic and textual images that are logically correlated to one another and methods of manufacturing absorbent articles with graphic and textual images. It is understood, however, that the invention is not limited to these specific embodiments and details, which are exemplary only. It further is understood that one of ordinary skill in the art, in light of known systems and methods, would appreciate the use of the invention for its intended purposes and benefits in any number of alternative embodiments, depending upon specific design and other needs.

As used herein, the terms “children's absorbent garment,” “children's absorbent article” or simply “article” or “garment” refer to devices worn by children that absorb and contain body fluids and other body exudates. More specifically, these terms refer to garments that are placed against or in proximity to the body of a wearer to absorb and contain the various exudates discharged from the body. A non-exhaustive list of examples of children's absorbent garments includes diapers, diaper covers, disposable diapers, training pants, pull-up pants, swimming pants, youth pants and nighttime training pants. Such garments may be intended to be discarded or partially discarded after a single use (“disposable” garments). Such garments may comprise essentially a single inseparable structure (“unitary” garments), or they may comprise replaceable inserts or other interchangeable parts.

The present invention may be used with all of the foregoing classes of children's absorbent garments, without limitation, whether disposable or otherwise. The embodiments described herein provide, as an exemplary structure, a pull-up style training pant for a toddler, however this is not intended to limit the claimed invention. The invention will be understood to encompass, without limitation, all classes and types of children's absorbent garments, including those described herein. Preferably, the absorbent core is thin in order to improve the comfort and appearance of a garment.

Throughout this description, the expressions “upper layer,” “lower layer,” “above” and “below,” which refer to the various components included in the absorbent core units of the invention (including the layers surrounding the absorbent core units) are used merely to describe the spatial relationship between the respective components. The upper layer or component “above” the other component need not always remain vertically above the core or component, and the lower layer or component “below” the other component need not always remain vertically below the core or component. Indeed, embodiments of the invention include various configurations whereby the core is folded in such a manner that the upper layer ultimately becomes the vertically highest and vertically lowest layer at the same time. Other configurations are contemplated within the context of the present invention.

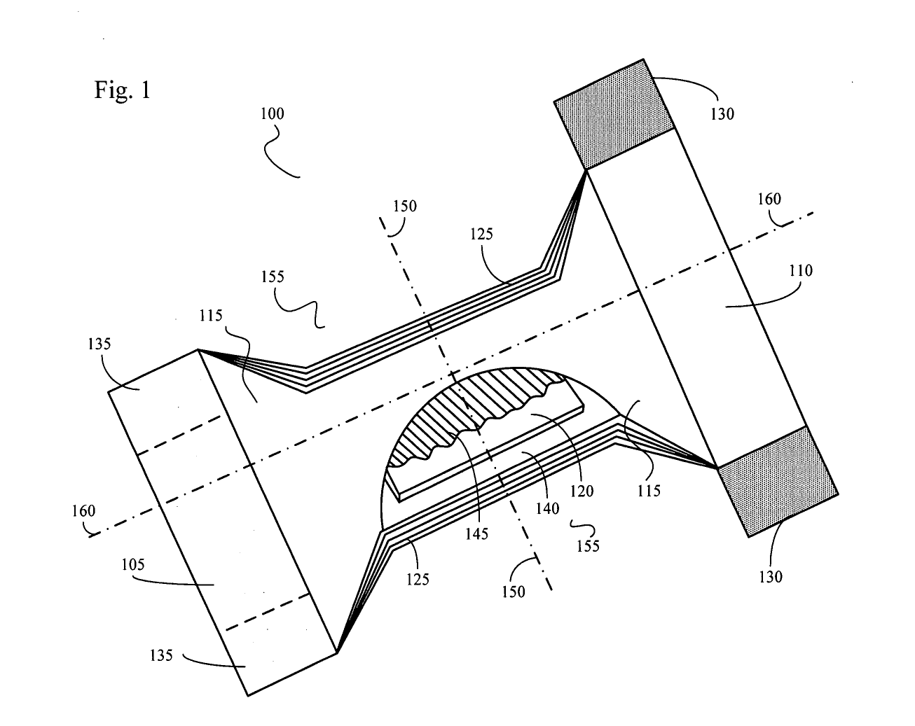

In addition, upper and lower layer refers to the ultimate configuration of the absorbent core as shown in FIG. 1.

The term “component” can refer, but is not limited, to designated selected regions, such as edges, corners, sides or the like; structural members, such as elastic strips, absorbent pads, stretchable layers or panels, layers of material, or the like; or a graphic.

Throughout this description, the term “disposed” and the expressions “disposed on,” “disposing on,” “disposed in,” “disposed between” and variations thereof are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the terms “top sheet” and “back sheet” denote the relationship of these materials or layers with respect to the absorbent core. It is understood that additional layers may be present between the absorbent core and the top sheet and back sheet, and that additional layers and other materials may be present on the side opposite the absorbent core from either the top sheet or the back sheet.

Throughout this description, the terms “graphic image” or “image graphic” are synonymous and refer to any graphic image which is printed on, embossed into or otherwise disposed so as to be visible from an outer facing surface of an absorbent article. Typically, though not necessarily, such graphic images are located on the front and optionally the rear facing outer surface in proximity to or aligned with the waist line of the absorbent article. The graphic image includes graphic images that are printed directly on a pre-printed poly or other component of the absorbent article, graphic images that are printed in-line during the article manufacturing process and component graphics that are printed on a separate roll that are either adhered to or otherwise affixed to the absorbent article during manufacture. The graphic image as described herein may depict an activity, state or condition and a textual graphic that is logically correlated to that activity, state or condition. In one embodiment, the graphic image depicts a character. The portion of the graphic depicting the character will be referred to herein as the “character graphic” and the portion of the graphic depicting the logically correlated textual description will be referred to as the “textual graphic.” Alternatively, the graphic image may depict a “state,” e.g., time of day such as nighttime, position of objects relative to one another, a character graphic's state of mind, a statement, a question or a “condition,” e.g., snowing, raining, hot, funny, wet, etc., along with a textual graphic logically correlated with the “state” or “condition.”

Throughout this description the term “logically correlated” refers to the relationship between an activity, state or condition that is depicted in the image graphic and a textual image that is descriptive of that activity, state or condition. That is to say, a child who is about to graduate from training pants to underwear would intuitively understand the relationship between the activity, state or condition illustrated in the image graphic and the textual graphic associated therewith. For example, the image graphic might depict one or more humanoid or anthropomorphic characters engaged in jumping rope, and the words “Jumping Rope” would appear next to the image graphic. As another example, an anthropomorphic character might be depicted lying in a bedtime scene including the stars and moon with the words “Bed Time” next to the image graphic.

Throughout this description the term “activity” in the context of character graphics will refer to an action being performed by a character in the graphic image. This may include physical activities such as running, walking, jumping, ice skating, riding, swimming, dancing, etc., or, may also include other non-physical activities such as reading, sleeping, bathing, painting, listening to music, singing, using the toilet, etc. Virtually any activity which can be illustrated through the use of humanoid or anthropomorphic characters with sufficient specificity so as to be readily discernable by a person viewing the character graphics may be used with this invention.

Throughout this description the term “character graphic” is used herein to refer to a graphic containing a humanoid or anthropomorphic image, and in particular an image having or suggesting human form or appearance which ascribes human motivations, characteristics or behavior to inanimate objects, animals, natural phenomena, cartoon characters, or the like. Ideally, the character graphic would be suitable for children's underwear and could be utilized to motivate children to wear the pants and use a potty or toilet. Suitable character graphics can include animals, people, inanimate objects, natural phenomena, cartoon characters, or the like that can or can not be provided with human features such as arms, legs, facial features of the like.

Throughout this description the term “state” is used herein to refer to a mode or condition of being, for example, the weather outside, the time of day, position of animate or inanimate objects relative to one another, and perhaps most importantly in the context of the preferred embodiments the character graphic's state of mind or awareness or the like, including statements made or questions asked in the textual graphic.

Throughout this description the term “condition” is used herein to refer to the influence of existing circumstances that create a scene. For example, a condition, could include but is not limited to the appearance of an image graphic, the health or well-being of a character depicted in an image graphic and the like, but could also include environmental conditions such as the weather.

The present invention relates generally to absorbent articles, and in particular to children's absorbent articles having a top sheet, a back sheet, an absorbent core disposed at least partially between the top sheet and the back sheet, and a graphic image disposed so as to be visible from one outer-facing surface of the back sheet, the graphic image depicting an activity, state or condition and a textual graphic wherein the textual graphic is logically correlated to the activity, state or condition depicted in the character graphic. In a preferred embodiment, the graphic image is disposed on a portion of the absorbent article corresponding to the front waist and, optionally, the rear waist of the wearer when the article is worn with a horizontal orientation. The absorbent core may be comprised of a central fibrous layer and one or more upper and/or lower layers, whereby the central fibrous layer is a fibrous structure comprised of natural or synthetic fibers and, optionally, super absorbent polymer (SAP), but may also be comprised of any suitable absorbent material.

The present invention also relates in general to a method of making an absorbent article that includes providing a top sheet material, a back sheet material, an absorbent core that contains at least one central fibrous layer comprised of a natural or synthetic fibrous material and SAP, and a graphic image that is visible from an outer-facing surface of the back sheet material. The absorbent core of the invention preferably is comprised of a central fibrous layer and one or more upper and/or lower layers, whereby the central fibrous layer is a fibrous structure comprised of natural or synthetic fibers and, optionally, SAP, but may also be comprised of any suitable absorbent material. The graphic image may be covered by one or more partially transparent layers. However, in a preferred embodiment, the graphic image will be disposed on the absorbent article so as to limit and more preferably minimize image occlusion by intervening layers.

The present invention also relates to a graphic image disposed so that it is visible from an outer facing surface of an absorbent article comprising an image graphic depicting an activity, state or condition and a textual graphic wherein the textual graphic is logically correlated to the activity, state or condition depicted in the image graphic. In a preferred embodiment, each graphic and textual image are one of a sequence of different graphic and textual images printed on a roll good material which can be individually separated and attached to an absorbent article in the absorbent article manufacturing process. In that manner, a variety pack of absorbent articles is provided whereby the package of absorbent articles contains two or more distinct image and textual graphics in the package of absorbent articles.

The present invention is premised in part on the theory that by providing a child with an absorbent article having a graphic image depicting a familiar and pleasing character activity, state or condition, along with a textual description of the activity, state or condition that is logically correlated to the image graphic, the child's mental development may be stimulated and engaged as part of the diapering or potty training process. For instance, the child will be more likely to commit to memory the name of the activity, the child's mental association between the activity and the word or phrase may be increased, the child's attitude toward the activity, state or condition may be enhanced, the child's recognition of letters may be improved and the child's behavior may be influenced by behavior illustrated by the activity, state or condition depicted along with textual description. Traditional graphic images on children's absorbent articles that do not employ graphic images logically correlated to textual graphics are less effective as educational and motivational aids to assist in overall learning as well as toilet training. These conventional graphic images fail to fully capitalize on the opportunity that care givers have to interact with children through the diapering and toilet training process. Absorbent articles including graphical objects and the word corresponding to the object are less effective at influencing the child's behaviors and do not fully stimulate his/her developing and impressionable mind.

The invention now will be described with reference to the attached drawings illustrating preferred embodiments of the invention. For clarity, features that appear in more than one Figure have the same reference number in each Figure. For sake of simplicity and ease of explanation, the invention will be discussed and illustrated in the context of pull-up type training pant articles. However, it should be appreciated by those of ordinary skill in the art, that the principles disclosed herein may be applied equally to any other type of children's absorbent article giving the term its broadest reasonable interpretation as defined herein.

Referring now to FIG. 1, a typical absorbent article 100 is illustrated in a partial cut away perspective view. The absorbent article depicted in FIG. 1 is an infant disposable diaper, however, as discussed above, this depiction is not intended to limit the invention. Those skilled in the art will appreciate that the invention will cover other types of absorbent articles as well. The absorbent article 100 of FIG. 1 is depicted in a generally flattened position, with the body-facing side facing up and the outer side facing down, and with the various elastic components depicted in the relaxed position with the effects of elasticity removed (typically relaxed elastics would cause the garment to bunch or contract around the locations of the elastics). In the flattened position, the absorbent article 100 has a generally hourglass configuration. Disposable diapers in particular will typically have either an hourglass, rectangular, or even T shape.

As used in this disclosure, the longitudinal axis, axis 160 in FIG. 1, of an absorbent article and/or absorbent core is the dimension corresponding to the front to rear axis of the wearer, and the transverse axis, 150 in FIG. 1, is the side-to-side axis of the wearer. The side to side axis 150 will generally provide a cross sectional view of the absorbent article and/or absorbent core.

The exemplary absorbent article 100 of FIG. 1 may include a top sheet 115, a back sheet 140 and an absorbent core 120 secured between at least portions of the top sheet 115 and back sheet 140. The top sheet layer 115 typically consists of a hydrophobic, yet fluid pervious web material that allows fluid to pass through to the absorbent core without absorption and that is relatively comfortable against the skin of the wearer. The back sheet layer 140 is typically comprised of a fluid impervious layer such as a poly-based film material which is typically coated with a thin external cloth-like layer designed to simulate a fabric material and de-emphasize the garment's plasticity. The top sheet layer 115 and the back sheet layer 140 may be constructed from a wide variety of materials known in the art. In the invention is not intended to be limited to any specific materials for these components. The top sheet layer 115 and back sheet layer 140 can be shaped and sized according to the requirements of each of the various types of absorbent garments, or to accommodate various user sizes. In an embodiment of the invention in which the absorbent article is a diaper or adult incontinence brief, the combination of the top sheet 115 and the back sheet 140 may have an hourglass shape, as seen in FIG. 1, or a rectangular, trapezoidal, “T” shape, or any other shape.

Due to the wide variety of backing and liner sheet constructions and materials currently available, the invention is not intended to be limited to any specific materials or constructions of these components. The back sheet 140 preferably is made from any suitable pliable liquid-impervious material known in the art. Typical back sheet materials include films of polyethylene, polypropylene, polyester, nylon, and polyvinal chloride and blends of these materials. For example, the back sheet can be made of a polyethylene film having a thickness in the range of 0.02-0.04 mm. The back sheet 140 may be pigmented with, for example, titanium dioxide, to provide the garment 100 with a pleasing color or to render the back sheet 140 opaque enough that exudates being contained by the garment 100 are not visible from outside the garment. In addition, the back sheet 140 may be formed in such a manner that it is opaque, for example, by using various inert components in the polymeric film and then biaxially stretching the film. Other back sheet materials will be readily apparent to those skilled in the art. The back sheet 140 preferably has sufficient liquid imperviousness to prevent any leakage of fluids. The required level of liquid imperviousness may vary between different locations on the garment 100.

The back sheet 140 may further comprise separate regions having different properties. In a preferred embodiment, portions of the back sheet 140 are air permeable to improve the breathability, and therefore comfort of the garment 100. The different regions may be formed by making the back sheet 140 of a composite of different sheet materials, chemical treatment, heat treatment, or other processes or methods known in the art. Some regions of the back sheet 140 may be fluid pervious. In various exemplary embodiments of the invention, the back sheet 140 is fluid impervious in the crotch region 155 but is fluid pervious in the waist regions 105 and 110. The back sheet 140 may also be made from a-laminate of overlaid sheet materials.

The moisture pervious top sheet 115 can be comprised of any suitable relatively liquid-pervious material known in the art that permits passage of liquid there through. Non-woven liner sheet materials are exemplary because such materials readily allow the passage of liquids to the underlying absorbent core 120. Examples of suitable liner sheet materials includes non-woven spun bond or carded webs of polypropylene, polyethylene, nylon, polyester and blends of these materials.

The back sheet 140 may be covered with a fibrous, non-woven fabric such as is disclosed, for example, in U.S. Pat. No. 4,646,362 issued to Heran et al., the disclosure of which is incorporated by reference in its entirety and in a manner consistent with this disclosure. Materials for such a fibrous outer liner include a spun-bonded non-woven web of synthetic fibers such as polypropylene, polyethylene or polyester fibers, a non-woven web of cellulosic and textile fibers, a spun-bonded non-woven web of synthetic fibers such as polypropylene, polyethylene or polyester fibers mixed with cellulosic, pulp fibers, or textile fibers, or melt blown thermoplastic fibers, such as macro fibers or micro fibers or polypropylene, polyethylene, polyester or other thermoplastic materials or mixtures of such thermoplastic macro fibers or micro fibers with cellulosic, pulp or textile fibers. Alternatively, the back sheet 140 may comprise three panels wherein a central poly back sheet panel is positioned closest to the absorbent core 120 while outboard non-woven breathable side back sheet panels are attached to the side edges of the central poly back sheet panel. Alternatively, the back sheet 140 may be formed from microporous poly cover stock for added breathability.

The layout of the absorbent article 100 is such that there is a front waist region 105, a rear waist region 110 and a crotch region 155 connecting the front waist region 105 and rear waist region 110. The labels front and rear are derived from their position on the wearer's body during use. The crotch region 155 may contain one or more leg elastics 125 and/or leg cuffs (not shown in drawing figure) which run the length of either edge of the tapered crotch region 155. The leg elastics 125 and/or leg cuffs provide a secure fit on the wearer and assist in trapping liquid and solid waste released by the wearer during use. The crotch region 155 is designed to receive and retain fluid through a fluid absorption and retention system. The fluid absorption and retention system consists of an absorbent core 120 located under the top sheet layer 115. Optionally, a fluid acquisition layer 145 may be located on a top surface of the absorbent core 120 to assist in absorption of fluid to the core. Typically, such a fluid acquisition layer 145 will have a higher acquisition rate than the material used in the core 120 to assist in pulling fluid away from the wearer. Underneath the absorbent core is the back sheet layer 140 which, as noted above, is typically constructed of a fluid impervious material. In order to expedite the manufacturing process, the top sheet layer 114, the absorbent core 120, the back sheet layer 140 and any acquisition layer 145 are joined into a single garment by joining multiple webs of stock materials which are held together with adhesives. Through such a process, several hundred units of absorbent garments may be produced in a single minute.

The absorbent core 120, including the optional fluid acquisition layer 145, can comprise one or more layers. In addition, one or more additional layers (not shown) may be disposed either under or over the absorbent core 120, such as between the absorbent core 120 and the back sheet layer 140 or between the absorbent core 120 and the top sheet layer 115. The additional layers may include a fluid transfer layer, a fluid handling layer, a storage layer, a wicking layer, a fluid distribution layer, and any other layer(s) known to those having ordinary skill in the art.

Although the absorbent core 120 depicted in FIG. 1 has a substantially rectangular shape, other shapes may be used such as an hour glass shape, or elliptical shape. The shape of the absorbent core 120 may be selected to maximize absorbency with a minimum amount of material. The absorbent core 120 may be affixed to the top sheet 115, the back sheet 140, or any other suitable part of the garment 100 by any method known in the art, in order to fix the absorbent core 120 in place. In addition to any individual layers within the absorbent core 120, the overall core 120 may be enclosed within a tissue wrapping, as disclosed in U.S. Pat. No. 6,068,620, the disclosure of which is hereby incorporated by reference in its entirety.

Though the absorbent core 120 depicted in FIG. 1 is shown as terminating prior to the either the front waist region 105 or the rear waist region 110, the absorbent core 120 may also extend into either of these portions. A typical absorbent core 120 will usually contain at least a central fibrous layer, and one or more other layers, such as, for example, a fibrous layer, a fluid management layer and a tissue layer. The central fibrous layer of the absorbent core 120 will comprise a fibrous structure. Central fibrous layers of this type generally are known in the art, and exemplary absorbent cores are described in U.S. Pat. Nos. 6,068,620 and 5,281,207, both issued to Chmielewski, and U.S. Pat. No. 5,863,288, issued to Baker, the disclosures of each of which are hereby incorporated by reference in their entirety in a manner consistent with this disclosure.

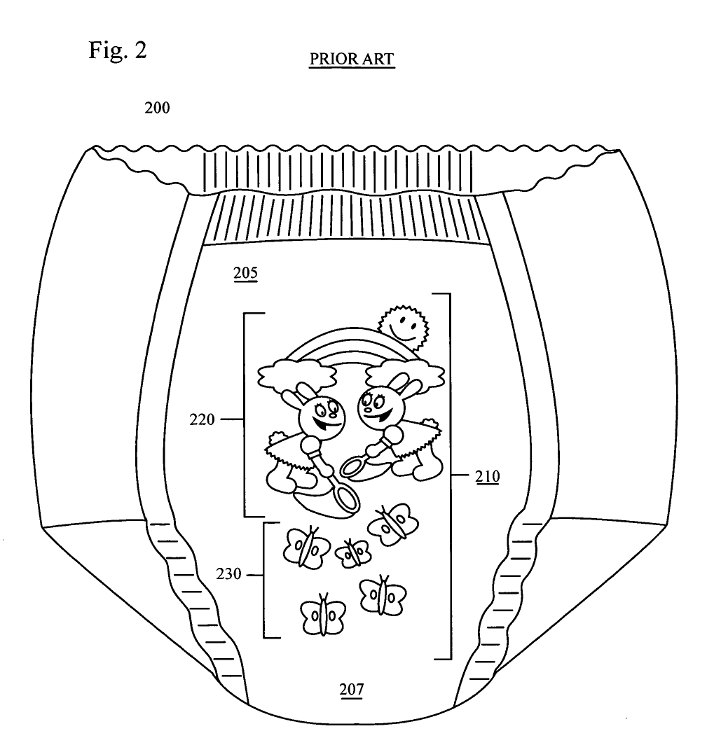

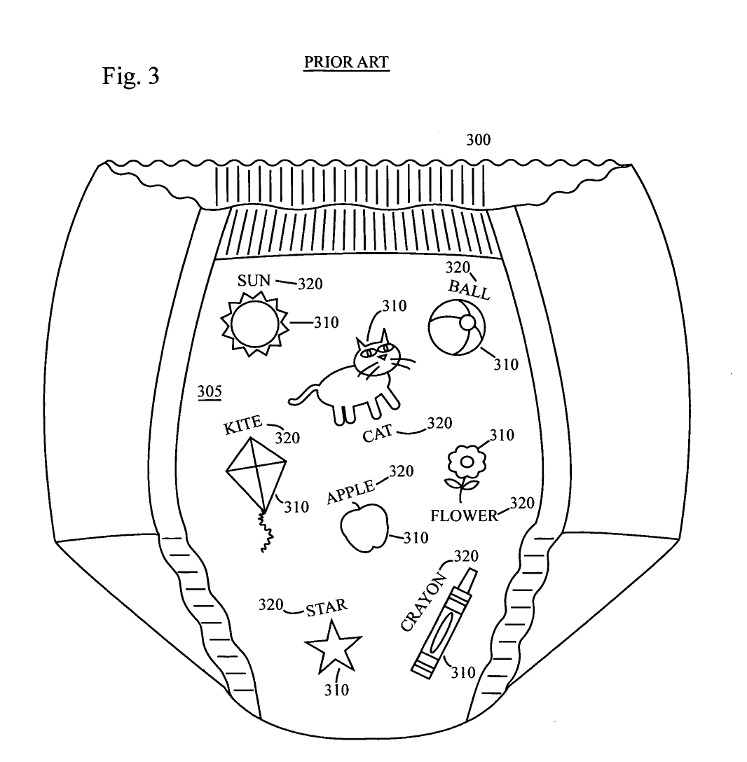

Referring now to FIGS. 2 and 3, these Figures illustrates disposable absorbent articles employing conventional graphic images as are known in the art. FIG. 2 illustrates an absorbent article 200 having a graphic image 210 comprised of a permanent graphic region 220 and an active graphic region 230 located on a front-facing side 205 of the absorbent article from the perspective of someone viewing the article head on. In the graphic image 210 of FIG. 2, the permanent graphic region 220 depicts a pair of humanoid or anthropomorphic rabbits holding butterfly nets and shown in a pleasing landscape environment that including clouds, a rainbow and the smiling sun. Typically, permanent graphics are located at or close to the front waist portion of the article and are usually registered using one or more of a variety of permanent or semi-permanent inks that, in contrast to active inks, are not intended to be activated by exposure to air, light or moisture. The active graphic region 230 depicts a group of butterflies printed on a crotch portion 207 of the absorbent article 200. Because the primary purpose of active graphics is wetness indication, they are generally located at the front crotch portion of the garment because it is likely to be the first to show signs of wetting. In the example shown in FIG. 2 the active graphics are normally apparent but become occluded when wet. However, other wetness indicating absorbent articles available may use inks that are become visible only after wetting. As with the disappearing type of graphics, this type of hidden graphic is also used for toilet training purposes to provide a feedback response to children after they have urinated in their diaper rather than in the toilet and a visual cue to the caregiver that the child's diaper needs to be changed.

The active graphics region 230 shown in FIG. 2 is an extension of the scene shown in the permanent graphics region 220. However, in other wetness indicating absorbent articles the active graphics region may be completely separate from and/or unrelated to the graphics shown in the permanent graphic region 220. For example, simple objects such as stars; smiley faces, etc., which are not an extension of the graphics depicted in the permanent graphic region 220 may be used.

The primary value of active graphics is as a visual cue to caregivers that a wet diaper needs changing. They usually can not be seen by children when they are wearing the absorbent article because of their location in the crotch region. They also provide some limited aesthetic value by adding more graphics to outside of the diaper which may enhance its appearance and influence purchase decisions. However, they are not an believed to be effective educational and/or motivation tool for children.

FIG. 3 illustrates another exemplary disposable absorbent article employing conventional graphic images. The absorbent article 300 depicted in FIG. 3 includes a variety of graphically illustrated objects 310, each with the word 320 corresponding to the objects 310 on the front-facing surface 305 of the absorbent article 300. The objects 310 shown in FIG. 3 include simple objects that are likely to be recognizable by a child of toilet training age, such as the sun, a star, a kite, a ball, a flower, a crayon, etc. While the graphics images employed in the conventional article of FIG. 3 may be useful for teaching word association between simple objects and their names, they are not illustrative of activities that children may be engaged in, nursery rhymes, the weather, a mood or feeling, thought or behavior, nor are they capable of suggesting or promoting specific behaviors or developing affinities for particular activities.

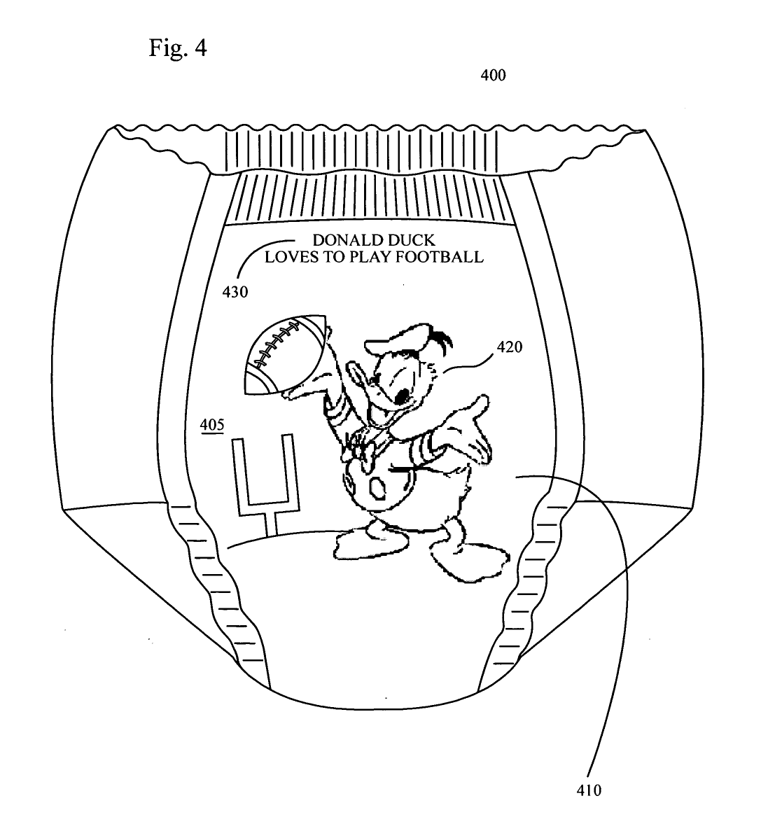

Referring now to FIGS. 4-9, each of these Figures illustrate absorbent articles employing graphic images according to various embodiments of this invention. In the absorbent article 400 of FIG. 4, the graphic image 410 includes a character image 420 and a logically correlated textual image 430 printed on the front facing surface 405 of the absorbent article 400. The character image 420 comprises an anthropomorphic character (Donald Duck®) depicted as playing football. The logically correlated textual image 430 reads, “Donald Duck loves to play football.” Although the entire phrase, “Donald Duck loves to play football,” is used in the exemplary embodiment of FIG. 4, the logically correlated textual image 430 may only state the activity “football,” or other permutations of playing football, such as, for example, “Throw the ball, Donald,” “Playing football is fun,” etc. Numerous different textual graphics may be used with the activity of “playing football” so long as they are sufficient to allow the caregiver to illustrate and describe to the child for whom the article is intended an example of what the activity of playing football “looks like” as well along with a corresponding logically correlated textual image. Furthermore, because in the image of the anthropomorphic character is shown as enjoying the activity, the child may develop positive associations with the activity of playing football in addition to learning to recognize the activity and the logically correlated expression, word or phrase corresponding to the textual image.

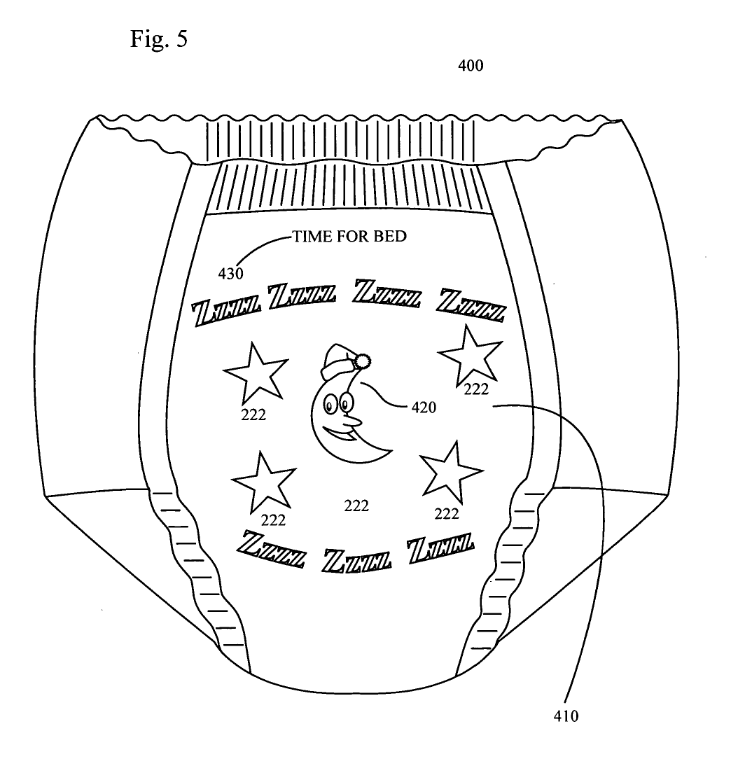

FIG. 5 is a perspective view of another pull-up style absorbent article 400 employing a graphic image 410 in accordance with various embodiments of this invention. In the embodiment illustrated in FIG. 5, the graphic image 410 is also comprised of a character graphic portion 420 and a logically correlated textual graphic portion 430. The character graphic portion 420 shown in this embodiment consists of an anthropomorphic moon dressed in a night cap, several stars, and several “Zzzz”s. The logically correlated textual image 430 reads “Time for bed.” The graphic image 400 in this example allows the caregiver to teach to the child things associated with bed time. For example, as shown in FIG. 5, this may include the moon and the stars, a night cap and catching “Zzzz”s. Also, because the moon is smiling, this may reduce anxiety and/or negative feelings the child has towards bed time. Moreover, the child is able to recognize words and phrases connoting bed time in association with logically correlated images that relate to the activity of going to bed. It should be appreciated that the textual graphic 430 of FIG. 4 may only name the activity, state or condition, i.e., “bedtime”, or may recite other permutations, such as, for example, “Good night,” or even a common childhood bed time nursery rhyme or bed time prayer without departing from the spirit or scope of the invention, so long as the textual graphic 430 is logically correlated to sleeping.

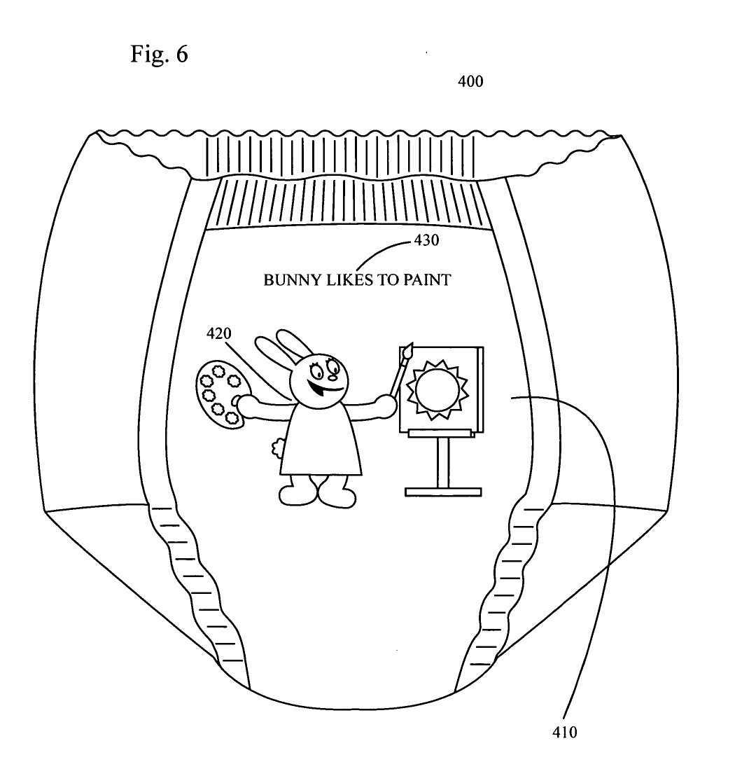

FIG. 6 is a perspective view of another pull-up style absorbent article 400 employing a graphic image 410 in accordance with various embodiments of this invention. In the embodiment illustrated in FIG. 6, the graphic image 410 includes a graphic image portion 420 consisting of an anthropomorphic rabbit painting a picture and a logically correlated textual graphic 430 that reads “Bunny likes to paint.” The graphic image 410 in this example allows the caregiver to teach to the child about the activity of painting. For example, as shown in FIG. 6, this may include using a paint brush, mixing board and easel. Also, because the anthropomorphic bunny rabbit is smiling, this may enhance the child's interest in painting. Moreover, the child is able to recognize the words or phrasing connoting painting in association with images that relate to the activity of painting. As with the exemplary embodiment illustrated in FIG. 4, the textual graphic 430 may only name the activity, i.e., “painting”, or may recite other permutations without departing from the spirit or scope of the invention.

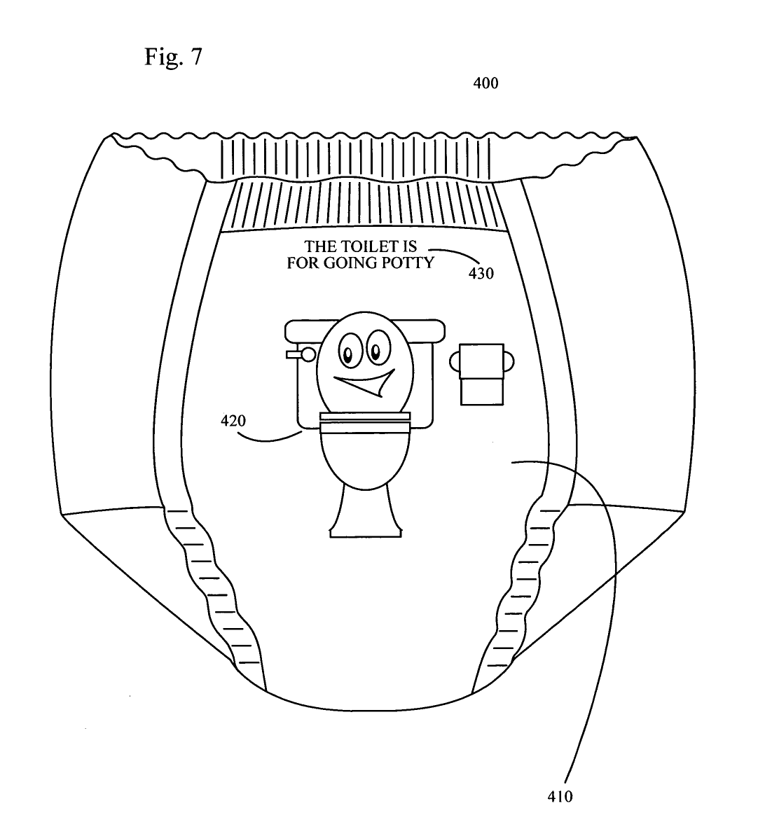

FIG. 7 is a perspective view of another pull-up style absorbent article 400 employing a graphic image 410 in accordance with various embodiments of this invention. In the embodiment illustrated in FIG. 7, the graphic image 410 is comprised of a character graphic portion 420 consisting of an anthropomorphic toilet and a logically correlated textual graphic 430 consisting of a motivational toilet training message such as, for example, “The toilet is for going potty.” The graphic image 410 in the embodiment shown in FIG. 7 is intended to motivate and encourage children to become toilet trained and to reduce anxiety associated with using the toilet. Furthermore, through the use of this graphic image 410, the child can learn to recognize the words and phrases connoting using the “toilet” and “going potty” and to associate them with positive feelings.

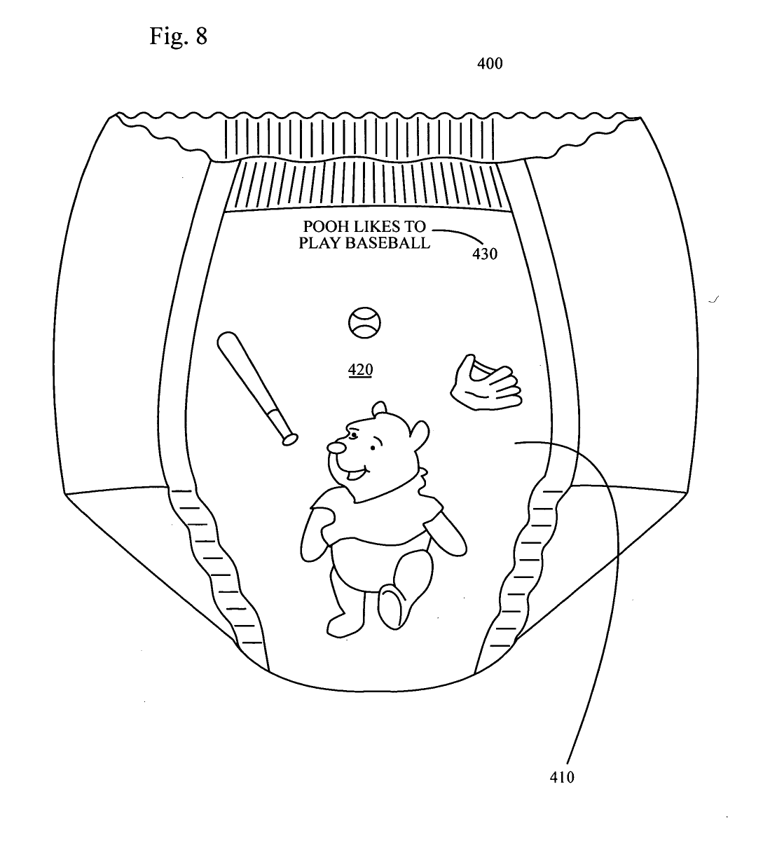

FIG. 8 is a perspective view of a further pull-up style absorbent article employing graphic images in accordance with additional preferred embodiments of this invention. The absorbent article 400 of FIG. 8 includes a graphic image portion 410 comprising an character graphic 420 depicting the popular children's character Winnie the Pooh® along with a baseball bat, ball and mitt and a logically correlated textual graphic 430 that reads “Pooh likes to play baseball.” Winnie the Pooh® is a character taken from the series of children's books written by A. A. Milne and illustrated by E. H. Shepard. Winnie the Pooh® is a registered trademark of the Walt Disney Company. Today, makers of children's products manufacture a variety of products including licensed images taken from the Winnie the Pooh® series of stories. Numerous cartoons, videos and movies feature these characters. Therefore, due to their familiarity, they are effective at entertaining and capturing the attention of children. In the embodiment illustrated in FIG. 8, a child can learn about the game of baseball from the combination of character graphics 420 and textual graphics 430.

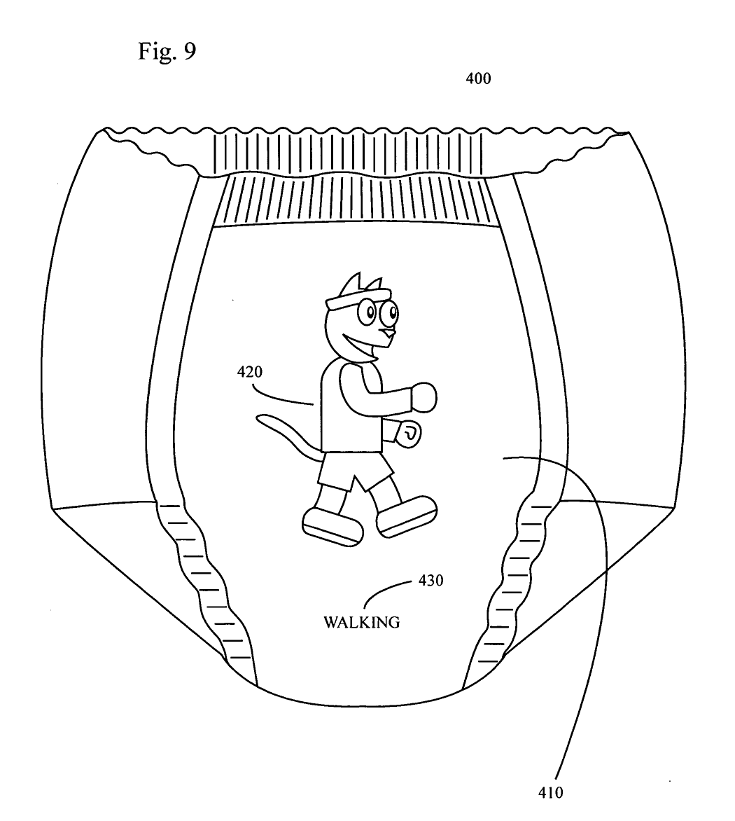

FIG. 9 is a perspective view of yet an additional pull-up style absorbent article employing graphic images in accordance with various embodiments of this invention. The absorbent article 400 of FIG. 9 includes a graphic image portion 410 comprising a character graphic 420 depicting an anthropomorphic cat in an athletic outfit and a logically correlated textual graphic 430 that reads “walking.” This graphic image allows a caregiver to teach a child about the activity of walking and to teach the child to recognize the word “walking.”

In each of the examples illustrated and described in FIGS. 4-9, absorbent articles are provided with graphic images that include a combination of character graphics and textual graphics wherein the textual graphic describes and is logically correlated to the activity, state or condition that the anthropomorphic character of the character graphic is depicted as engaged in. By using a character graphic which depicts a familiar and/or pleasing character engaged in a specific activity, state or condition and a textual graphic which describes the activity, state or condition, either with a single word, a phrase, or an expression, it is believed that a greater educational benefit is derived by the child and enhanced interaction between caregiver and child may be facilitated. Furthermore, associations between activities and words may be created in the children's minds and they may even be induced and/or encouraged to participate in these activities because of this association.

Though FIGS. 4, 6, 8 and 9 illustrate anthropomorphic characters as humanized animal characters or humanoids, as evidenced by FIG. 5 and 7, the anthropomorphic character may also be an inanimate object which has been illustrated to include humanistic features such as a face, arms, legs, clothing, etc.

It should be noted that though active graphics were discussed in the context of prior art-type graphic images, that the graphic images according to various embodiments of this invention may employ only permanent/semi-permanent graphics, only active graphics, or combinations of permanent/semi-permanent and active graphics. The specific properties of the dyes and chemicals used to create graphics is not relevant to the new and useful features of the embodiments of this invention.

### EXAMPLES

While the invention has been described in connection with the preferred embodiments, it is readily contemplated that there are many different combinations of images graphics and logically correlated textual graphics within the scope of the preferred embodiments. The following examples are but a few of the many different combinations of image graphics and logically correlated textual graphics used in absorbent garments within the scope of the claimed invention.

### Example 1

The first example contemplates the use of nursery rhymes. Children are often very familiar with nursery rhymes, the characters in the nursery rhymes, and the associated story line in nursery rhymes. The table below provides but a few examples of the possible combinations of image graphics depicting an activity, state or condition and a textual graphic logically correlated to the image graphic. For example, a image graphic might depict a man snoring with raindrops falling along with a logically correlated textual graphic reciting that “It's raining, it's poring, the old man is snoring.” Another example where the invention according to the preferred embodiments might incorporate a nursery rhyme could include a image graphic of an anthropomorphic egg sitting on a wall along with the textual graphic reciting “Humpty Dumpty sat on a wall.”

Example 1: Nursery Rhymes

| | |
|---|---|
| Image Graphic Depicting | Textual Graphic Logically |
| Activity, State or Condition | Correlated to Image Graphic |
| Graphic of raindrops with man | “It's raining, it's pouring, the old |
| snoring | man is snoring” |
| Graphic of spider climbing up | “The itsy bitsy spider climbed up |
| water spout | the water spout” |
| Graphic of egg sitting on a wall | “Humpty Dumpty sat on a wall” |
| Graphic of boy and girl running | “Jack and Jill went up a hill” |
| up a hill | |
| Graphic of girl and lamb | “Mary had a little lamb” |
| Graphic of shining stars | “Twinkle, twinkle little star” |
| Graphic of girl and sheep | “Little Bo peep has lost her sheep” |

### Example 2

Another combination of image and logically correlated textual graphics within the scope of the preferred embodiments might include image graphics that depict generically an activity, state or condition along with a logically correlated textual graphic. For example, an image graphic might depict two or more children playing Patty-Cake, along with the logically correlated textual graphic reading “Patty-cake.” This would obviously depict the two children engaged in the well know children's game known as “Patty-Cake.” Alternatively, a generic image graphic might depict an environmental scene or condition such as snow along with a textual graphic describing the state or condition that it is “snowing” outside. Still further yet, a generic image graphic may depict a child sledding, along with the textual graphic “Winter is fun.” Still further yet, a generic image graphic might depict a small school of fish swimming, along with the textual graphic asking “How many fish can you count?” Further yet, a generic image graphic might object various numbers or letters of the alphabet along with the logically correlated textual graphic asking “What numbers/letters do you see?” These are but a few examples of the types of generic images and textual graphics associations that may be included on an absorbent garment within the scope of the preferred embodiments. More are provided in the table below.

Example 2: Generic Pictorial and Textual Graphic Associations

| Image Graphic Depicting | Textual Graphic Logically |
|---|---|
| Activity, State or Condition | Correlated to Image Graphic |
| Graphic of two or more children | “Patty-cake” |
| playing patty-cake | |
| Graphic of rain drops | “Raining” |
| Graphic of child sleeping | “Sleeping” |
| Graphic of child going to bed | “Time to go to bed” |
| Graphic of snow | “Snowing” |
| Graphic of child sledding | “Winter is fun” |
| Graphic of American flag and | “Fireworks are fun” |
| fireworks | |
| Graphic of fish swimming | “How man fish can you count” |
| Graphic of girls jumping rope | “How many times can you jump” |
| Graphic of a birds flying | “Birds like to fly” |
| Graphic of airplane flying | “Flying above the clouds” |

### Example 3

Another absorbent garment within the scope of the preferred embodiments includes graphics depicting well known cartoon characters engaged in some sort of activity, state or condition along with a logically correlated textual graphic. For example an image graphic of Kermit the Frog® taking a bath with the logically correlated textual graphic reading “Frogs love water.” Yet another example of a cartoon character in a graphic image according to preferred embodiments may include a image graphic of Winnie the Pooh® eating honey with the logically correlated textual graphic “Bears love honey.” Still further yet, an image graphic of Cinderella wearing a glass slipper might be associated with a logically correlated textual graphic reading “The clock is about to strike midnight.” These are but a few of the image graphics and textual graphics that may be logically correlated using cartoon characters in accordance with the preferred embodiments.

Example 3: Cartoon Characters

| Image Graphic Depicting | Textual Graphic Logically |
|---|---|
| Activity, State or Condition | Correlated to Image Graphic |
| Graphic of Kermit the Frog ® | “Frogs love water” |
| taking a bath | |
| Graphic of Winnie the Pooh ® | “Bears love honey” |
| eating honey | |
| Graphic of Cinderella wearing a | “The clock is about to strike |
| glass slipper | midnight” |
| Graphic of Ariel swimming and | “Singing is fun” |
| singing | |
| Graphic of Berenstain Bears ® | “I love to picnic” |
| picnicking | |
| Graphic of Cookie Monster ® | “Cookie Monster loves cookies” |
| eating cookies | |
| Graphic of Blue's Clues ® | “I found a clue” |
| searching | |
| Graphic of Dora the Explorer ® on | “Animals are our friends” |
| a Safari | |

While the foregoing description includes many details and specificities, it is to be understood that these have been included for purposes of explanation only, and are not to be interpreted as limitations. Many modifications and equivalent substitutions to the embodiments described above can be made without departing from the spirit and scope of the invention.

1. An absorbent article comprising:
a top sheet layer;
a back sheet layer;
an absorbent core provided at least partially between the top sheet layer and the bottom sheet layer; and
an image graphic visible on the absorbent article, the image graphic depicting an activity, state or condition and a textual graphic wherein the textual graphic is logically correlated to the activity, state or condition depicted in the image graphic.

2. The absorbent article according to claim 1, wherein the image graphic depicts a character performing an activity and the textual graphic is descriptive of the activity.

3. The absorbent article according to claim 2, wherein the character is depicted performing an activity to be performed by a wearer of the absorbent article.

4. The absorbent article according to claim 2, wherein the character is an anthropomorphic or humanoid character.

5. The absorbent article according to claim 1, wherein the textual graphic is selected from the group consisting of a single word, multiple words, a phrase, a nursery rhyme or portion thereof, a command, a question, a formal name, an expression and combinations thereof.

6. The absorbent article according to claim 1, wherein the image graphic and textual graphic correspond to an age of the intended wearer of the absorbent article.

7. The absorbent article according to claim 6, wherein the age of the intended wearer of the absorbent article corresponds to a size of the absorbent article.

8. The absorbent article according to claim 6, wherein the age of the intended wearer of the absorbent article corresponds to a style of the absorbent article.

9. The absorbent article according to claim 1, wherein a style of the article is selected from the group consisting of an infant diaper, a toddler diaper, a youth training pant, a pull-up style article, a children's nighttime training pant, a swimming-only article, and combinations thereof.

10. The absorbent article according to claim 9, wherein the image graphic and the textual graphic correspond by gender to activities associated with a wearer of one or more of the styles of articles.

11. The absorbent article according to claim 1, wherein the image graphic is applied directly on an inward or outward facing portion of the back sheet layer.

12. The absorbent article according to claim 1, wherein the image graphic is applied on another layer which is attached to an outward facing portion of the back sheet layer.

13. A graphic image disposed so as to be visible on an outer facing surface of an absorbent article comprising:
an image graphic depicting an activity, state or condition and a textual graphic wherein the textual graphic is logically correlated to the activity, state or condition depicted in the image graphic.

14. The absorbent article according to claim 13, wherein the image graphic depicts a character performing an activity and the textual graphic is descriptive of the activity.

15. The absorbent article according to claim 14, wherein the character is depicted performing an activity to be performed by a wearer of the absorbent article.

16. The absorbent article according to claim 14, wherein the character is an anthropomorphic or humanoid character.

17. The absorbent article according to claim 13, wherein the textual graphic is selected from the group consisting of a single word, multiple words, a phrase, a command, a question, a formal name, an expression and combinations thereof.

18. The absorbent article according to claim 13, wherein the image graphic and textual graphic corresponds to an age of an intended wearer of the absorbent article.

19. The absorbent article according to claim 18, wherein the age of the intended wearer of the absorbent article corresponds to a size of the absorbent article.

20. The absorbent article according to claim 18, wherein the age of the intended wearer of the absorbent article corresponds to a style of the absorbent article.

21. The absorbent article according to claim 13, wherein a style of the article is selected from the group consisting of an infant diaper, a toddler diaper, a youth training pant, a pull-up style article, a children's nighttime training pant, a swimming-only article, and mixtures thereof.

22. The absorbent article according to claim 21, wherein the image graphic and textual graphic correspond to an activity associated with a wearer of one or more of the styles of articles.

23. The absorbent article according to claim 13, wherein the graphic image is applied directly on an inward or outward facing portion of the absorbent article.

24. The absorbent article according to claim 13, wherein the image graphic is applied on a first layer and the first layer is attached to the outward facing portion of a second layer in the absorbent article.

25. A method of manufacturing an absorbent article having an image graphic, comprising the steps of:
registering a graphic so as to be visible on an outer facing surface of the absorbent article, said graphic comprising an image graphic depicting an activity, state or condition and a textual graphic wherein the textual graphic is logically correlated to the activity, state or condition depicted in the image graphic.

26. The method according to claim 25, wherein the image graphic comprises a character performing an activity and a textual graphic is selected from the group consisting of a single word, multiple words, a phrase, a nursery rhyme or portion thereof, a command, a question, a formal name, an expression and combinations thereof.

27. The method according to claim 26, wherein the image graphic comprises a character performing an activity to be performed by a wearer of the absorbent article.

28. The method according to claim 26, wherein the character is an anthropomorphic character.

29. The method according to claim 25, wherein registering a textual graphic comprises registering a textual graphic selected from the group consisting of a single word, multiple words, a phrase, a nursery rhyme or portion thereof, a command, a sentence, a formal name, and combinations thereof.

30. The method according to claim 25, wherein registering the image comprises registering an image graphic having an intellectual complexity corresponding to an age of an intended wearer of the absorbent article.

31. The method according to claim 30, wherein the age of an intended wearer of the absorbent article corresponds to a size of the absorbent article.

32. The method according to claim 30, wherein the age of the intended wearer of the absorbent article corresponds to a style of the absorbent article.

33. The method according to claim 25, wherein a style of the article is selected from the group consisting of an infant diaper, a toddler diaper, a youth training pant, a pull-up style article, a children's nighttime training pant, a swimming-only article, and mixtures thereof.

34. The method according to claim 33, wherein registering the graphic comprises registering a character graphic and a textual graphic corresponding to activities associated with a wearer of one or more of the styles of articles.

35. The method according to claim 25, wherein registering the graphic on an outer facing surface of the absorbent article comprises applying the graphic directly on an inward or outward facing portion of a backsheet in the absorbent article.

36. The method according to claim 25, wherein registering the graphic on an outer facing surface of the absorbent article comprises applying the graphic on a first layer and attaching the first layer to an outward facing portion of a second layer in the absorbent article.
